(19) United States
(12) Patent Application Publication
(10) Pub. No.: US 2004/0241688 A1
(43) Pub. Date: Dec. 2, 2004

(54) Human tissue specific drug screening procedure

(76) Inventor: Cuneyt Bukusoglu, Norwood, MA (US)

Correspondence Address: BROWN, RUDNICK, BERLACK & ISRAELS, LLP., BOX IP, 18TH FLOOR, ONE FINANCIAL CENTER, BOSTON, MA, 02111, US

(21) Appl. No.: 10/484,286

(22) PCT Filed: Jul. 18, 2002
(86) PCT No.: PCT/US02/23138

Related U.S. Application Data
(60) Provisional application No. 60/307,062, filed on Jul. 19, 2001.

(51) Int. Cl. C12Q 1/68; G01N 33/53; G01N 33/558

(52) U.S. Cl. 435/6; 435/7.1; 436/514

(57) ABSTRACT

A method of using tissue cartridges containing one or more tissues samples in configuration allowing screening of drug candidates against normal or known disease states. The inventive method generates binding information for multiple drug-human tissue sections. This binding information helps identify drug candidates having specific binding characteristics allowing for selection of potential drug candidates having specific binding characteristics allowing for selection of potential drug candidates that have the desired binding qualities. The ability to understand binding characteristics allows drug discovery methods that reduce potential side effects.

### RELATED APPLICATIONS

This application claims the priority filing benefit of U.S. provisional patent application No. 60/307,062, filed on Jul. 19, 2001, which is incorporated in its entirety by reference.

### FIELD OF INVENTION

This invention is drawn to a procedure to screen a library of drug candidates on human or animal tissue specimens. The inventive procedure identifies affinity of compounds on tissue sections quickly and accurately. In one embodiment tissue sections are placed within tissue cartridges and a library of compounds are applied to the tissue sections. Compounds binding to tissue are eluted. Eluted compounds are analyzed to determine their affinity to target tissues.

### BACKGROUND OF INVENTION

There is currently a need in drug discovery and development and in general biological research for methods and apparatus for accurately performing tissue based assays. Although cell-based assays are advantageously employed for assessing the biological activity of chemical compounds and mechanism-of-action of new biological targets, there are no or limited current methods for employing multiplex-assays that are tissue based. In addition, there is a need to quickly and inexpensively screen large numbers of chemical compounds for their ability to bind to target tissues. This need has arisen in the pharmaceutical industry where it is common to test chemical compounds for activity against a variety of biochemical targets, for example, receptors, enzymes and nucleic acids. These chemical compounds are collected in large libraries, sometimes exceeding one million distinct compounds

Life science research has undergone a transition in recent years to large-scale experimentation, where drug discovery programs can require hundreds or thousands of measurements involving the consumption of significant time and resources. Many of these resources are consumed in efforts to focus the specificity and sensitivity of lead compounds. In addition, many programs are aborted after decades of costly yet fruitless efforts to limit side effects or toxicity of candidate drugs. Accordingly, tools that abbreviate the research and discovery phase of drug development are desirable.

In their search for new drugs, pharmaceutical researchers test many chemical compounds to determine whether they interact with drug targets. These researchers typically have large collections of chemical compounds to test against potential drug targets. Recently pharmaceutical researchers have been vastly expanding the size of compound collections they use to screen against new drug targets. As a result, researchers require new laboratory technologies capable of screening increasingly large compound collections against an increasing number of drug targets in a cost-effective, automated and rapid manner.

To screen a compound collection against a new drug target, a researcher must develop a test, or assay, for measuring whether particular chemical compounds in the library interact with the drug target in a certain manner. The type of assay selected depends on the drug target under investigation and the type of information being sought. Researchers design some assays to measure whether and how tightly a compound binds to a drug target, such as the binding of a drug to a protein. Other assays are designed to measure whether and to what degree a compound reduces the biological activity of a drug target, such as the activity of an enzyme. In other cases, researchers test compound collections against living cells and measure a particular cellular response, such as a change in expression level of one or more genes or the binding specificity of the drug candidate to a disease state. Unfortunately, the ability of pharmaceutical researchers to assess these compounds in an affinity based tissue binding assay is limited.

Current assay development methods are time consuming, taking from weeks to months, and are labor intensive, largely due to the need to measure a particular molecule within a complex mixture. In addition, current technologies for performing assays provide only a fraction of the information needed for selecting potential drug candidates. These current assays are primarily based upon cellular components rather than intact human tissue samples. Existing detection methods also typically require preparation of reagents in a highly purified form that requires additional expense, time and labor. These expensive reagents are routinely discarded as part of current assay protocols.

Secondary screening of drug candidates involves performing a variety of measurements on hits identified in a primary screen. While the number of compounds under investigation is smaller than in primary screening, the number and diversity of measurements performed on each compound is much larger. The purpose of these measurements is to verify and further characterize the biological activity of each hit. For example, researchers may test each hit against the drug target at different concentrations to determine its potency. Also, each hit may be tested against multiple enzymes to identify activity against any of these enzymes. Current function-based technologies typically measure only a single data point at a time, such as the effect of one compound on the activity of a particular enzyme, thus generating limited data according to known functional criteria.

Among current binding-based technologies “Phage-Display Procedure” is one method that allows screening of multiple phage particles on target samples such as pure proteins on solid surfaces or live cells in microwell plate formats. This procedure usually includes clearing and pre absorption, nonspecific particles, binding to specific targets, eluting and identifying specifically bound particles. Unfortunately, phage display procedures are limited to what they can express and display on surfaces (mostly small peptides). Also, screening steps are done by utilization of whole-phage particles which are larger structures as compared to small peptide sequences displayed on their surfaces.

Existing immunodiagnostic testing typically involves sophisticated instrumentation and multi-step protocols including sample dilution, variable incubation times and wash steps. Substantially all immunodiagnostic tests today are performed in centralized laboratories on complex instruments operated by skilled technicians. Unfortunately, despite the complexity of these instruments, the recovery and analysis of the flow through within these complex instruments is not possible.

Thus, there is a need for improved (e.g., faster and less expensive) methods for characterizing activities and targets of drugs based on binding characteristics of drug candidates upon tissue samples. The present invention provides methods for rapidly characterizing the specificity of the binding of candidate drugs to various tissue disease states.

### SUMMARY OF INVENTION

The present invention utilizes human tissue samples in a cartridge based system to screen drug candidates based upon binding of drug candidates on tissue samples. These human tissue samples, which are available from tissue block repositories, range from normal tissue samples to tissue representing all phases of disease development.

The method according to the invention utilizes a tissue cartridge containing one or more tissue samples in a configuration allowing screening of drug candidates against normal or known disease states. The inventive method generates binding information for multiple drug-human tissue sections. This binding information helps identify drug candidates having specific binding characteristics allowing for selection of potential drug candidates having desired binding qualities. The ability to understand binding characteristics facilitates drug discovery methods that reduce potential side effects.

The inventive method involves clearing, binding and elution cycles to screen drug candidates. According to the invention, a number of drug candidates designed to be disease-state specific are mixed with a pre-dilution buffer and applied to a series of pre-determined human tissue sections for pre-clearing purposes. The pre-dilution buffer is comprised of various components to mimic conditions a compound would encounter within a route of administration. These pre-clearing sections represent un-desired targets such as normal tissue samples. The pre-clearing procedure excludes candidates that bind to normal tissue samples.

According to the invention, during pre-clearing, drug candidates are applied to normal tissue samples contained within a tissue cartridge. Drug candidates that do not bind to normal tissue samples are referred to as “flow-through” molecules. Those molecules that bind to normal tissues are eliminated as potential drug candidates. The flow-through molecules are collected and identified (when needed) and applied to tissue cartridges containing tissue samples having a targeted disease state and are also applied once again to normal tissue samples. Flow-through molecules unbound to targeted tissue samples are eliminated as drug candidates and those that bind to normal tissue samples are also eliminated. Molecules binding to specific targeted tissues are eluted from the tissue sample by pre-determined elution buffers. The elution is analyzed and molecules having binding capability are detected and identified.

The eluted drug candidates' binding affinity and specificity may be further improved by a maturation process involving additional cycles of clearing, binding and elution. Eluted molecules may be further applied in parallel to both normal and disease state tissues from various organs of the human body. Tissue cartridges according to the invention, are configured in a manner allowing application of reagents and molecules to tissue sample with recapture of the applied matter. The inventive tissue cartridges allow for a closed system offering the following advantages: lack of dilution (due to pumping & replacement action), elimination of evaporation, recycling of reagent, incubation at a specified temperature, recovery of added reagent and recovery of bound material.

The tissue cartridge according to the invention is fabricated from various plastics or other materials known within the art and contains an inlet and outlet port. The cartridge allows for the insertion of at least one plate or membrane containing tissue samples and has a sealing means allowing a closed system. The cartridge has an interior configuration to prevent pooling, yet allows re-capture of reagent or applied material. Heating elements may be contained within the cartridge allowing for incubation at desired temperatures.

It is an object of the present invention to provide means by which the binding affinity of compounds to tissue samples can be determined.

A further object of the present invention is to provide an automatic method for pre-screening the ability of a molecule to bind to predetermined tissue.

Another object of the invention is to identify novel and specific targets in certain tissues and diseases.

It is yet another object of the present invention to identify molecules exhibiting multiple tissue targeting suggesting potential side effects and toxicological effects or additional pharmacological benefits.

It is another object of the invention to identify molecules having the highest specific tissue targeting. The objective is to achieve specific binding in target tissues while eliminating or controlling nonspecific or low-affinity binding target sites

### BRIEF DESCRIPTION OF THE DRAWINGS

The foregoing and other features and advantages of the present invention will be more fully understood from the following detailed description of illustrative embodiments, taken in conjunction with the accompanying drawings in which:

FIG. 1 is a diagrammatic representation of the tissue cartridge according to the invention;

FIG. 2 is a schematic of the inventive indirect method; and

FIG. 3 is a schematic of the inventive direct method.

### DETAILED DESCRIPTION OF THE INVENTION

The present invention relates to a method of screening drug candidates within drug libraries based upon binding of the drug candidates on tissue samples. The inventive method utilizes tissue cartridges to assess the binding of drug candidates to target tissue. The tissue cartridges according to the invention allow for tissue based affinity chromatography (“TBAC”).

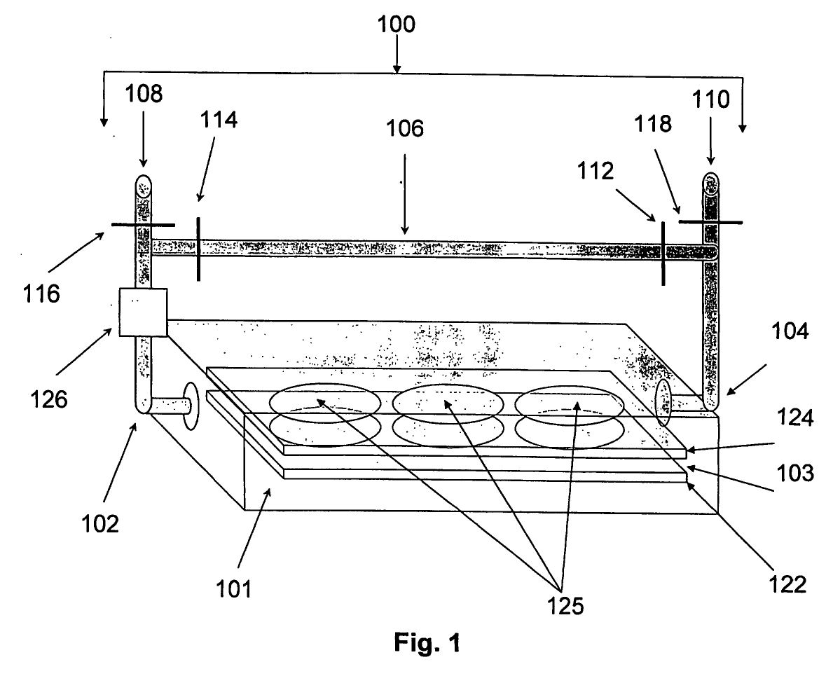

As shown in FIG. 1, the inventive tissue cartridge 100 has a sealable chamber 101 having an inlet port 102 and an outlet port 104. Connected to the inlet port 102 is an inlet channel 108. Connected to the outlet port 104 is an outlet channel 110. The inlet channel 108 contains an inlet valve 116 and the outlet channel 110 contains an outlet valve 118. The inlet channel 108 and the outlet channel 110 are connected to each other by a connecting channel 106. The connecting channel 106 contains a first connecting channel valve 112 and a second connecting channel valve 114. Within the inlet channel 108 a fluidic pump 126 is positioned. After fluid introduction via the inlet channel 108 and the closing of the inlet valve 116 and the outlet valve 118 and the opening of the first connecting channel valve 112 and the second connecting channel valve 114 the tissue cartridge 100 is a closed system. The closed system allows for the recycling of fluid through the tissue cartridge via the fluidic pump 126. The closing of the first channel valve 112 and the opening of the outlet valve 118 allow for the recovery of fluid circulated through the tissue cartridge 100.

It is contemplated within the scope of this invention that the inventive tissue cartridge 100 can be in fluid communication with each other forming a series of tissue cartridges. This series of tissue cartridges is formed by connecting the outlet channel 110 of one tissue cartridge to the inlet channel 108 of another tissue cartridge. It is contemplated within the scope of this invention that multiple cartridges can be connected together to form a fluidic tissue cartridge system.

The sealable chamber 101 has an inner chamber 103 having a volume of approximately 25 to 1000 μl. The inner chamber 103 houses a first plate 122 and a second plate 124. It is contemplated within the scope of the invention that the inner chamber 103 may contain additional plates. The first plate 122 and second plate 124 are housed within the inner chamber 103 so that all plate surfaces are exposed within the inner chamber 103. The first plate 122 and the second plate 124 are standard microscope slides. It is contemplated within the scope of the invention that the plates can be any solid surface or membrane known within the art. Each plate 122, 124 holds one or more tissue samples 125.

The inner chamber 101 may further contain a heating element (not shown). The heating element within one illustrative embodiment is in the form of resistive ink printed on the interior of the sealable chamber 101. In a alternative illustrative embodiment the heating element is in the form of a resistive element contained within the inner chamber 101. The sealable tissue cartridge 101 houses a thermocouple (not shown) that monitors the temperature of the inner chamber 102 controlling heating element. It is contemplated within the scope of the invention that the temperature of the tissue cartridge 100 can be controlled by the application of heat or cold to the exterior of the sealable tissue chamber 101.

The tissue cartridge 100 according to the invention can be manufactured by methods known to those skilled in the art. Sample plates, sealable tissue chambers and channel composition will be a function of structural requirements, manufacturing processes, and reagent compatibility/chemical resistance properties. The choice of materials will depend on a number of factors such as ease in manufacturing and inertness to fluids that will flow through and on sample plates, sealable tissue chambers and channels, as is known to those skilled in the art. Specifically, sample plates, sealable tissue chambers and channels are provided that are made from inorganic crystalline or amorphous materials, e.g. silicon, silica, quartz, inert metals, or from organic materials such as plastics, for example, poly(methyl methacrylate) (PMMA), acetonitrile-butadiene-styrene (ABS), polycarbonate, polyethylene, polystyrene, polyolefins, polypropylene, polyphenylene sulphide (PPS), PEEK, and metallocene. Sample plates, sealable tissue chambers, and channels according to the invention can be fabricated from thermoplastics such as polyethylene, polypropylene, methylmethacrylates, polycarbonates, and certain Teflons, among others, due to their ease of molding, stamping and milling. Alternatively, sample plates, sealable tissue chambers and channels can be made of silica, glass, quartz or inert metal.

A drug library of interest contained within a dilution buffer is applied to target tissue contained within the inventive tissue cartridge 100, incubated, washed and eluted. It is contemplated within the scope of the invention that drug libraries may be composed of peptides (chemical synthesis, phage-display, flagella display), small molecules, proteins, carbohydrates, lipids, steroid, and any other form of inorganic or organic molecule or their polymers.

The library of compounds are diluted in dilution buffers before tissue application. These dilution buffers allow a compound to be interacted with a tissue sample in conditions that mimic in-vivo conditions. Therefore, these dilution buffers will be composed of two main components a solution and protein that are buffered to a specific pH. The solution may be composed of phosphate buffered saline (PBS), saline, balanced salt solution (BSS) or selected mixture of serum components (SMSC) (e.g. amino acids, vitamins, ions, metabolites, lipids, carbohydrates, etc). SMSC may be composed of certain components to reflect pathological conditions such as high glucose for diabetes. The protein may include blood, serum or plasma at approximately 0.1-50% (v/v). The specific pH of dilution buffers may reflect arterial blood (7.38-7.44) or venous blood (7.76-7.41) or venous serum/plasma (7.35-7.45). Under certain conditions where maximum protein effect is needed, dilution buffers may be composed of 100% blood, serum or plasma. Other dilution buffers known in the art may also be used.

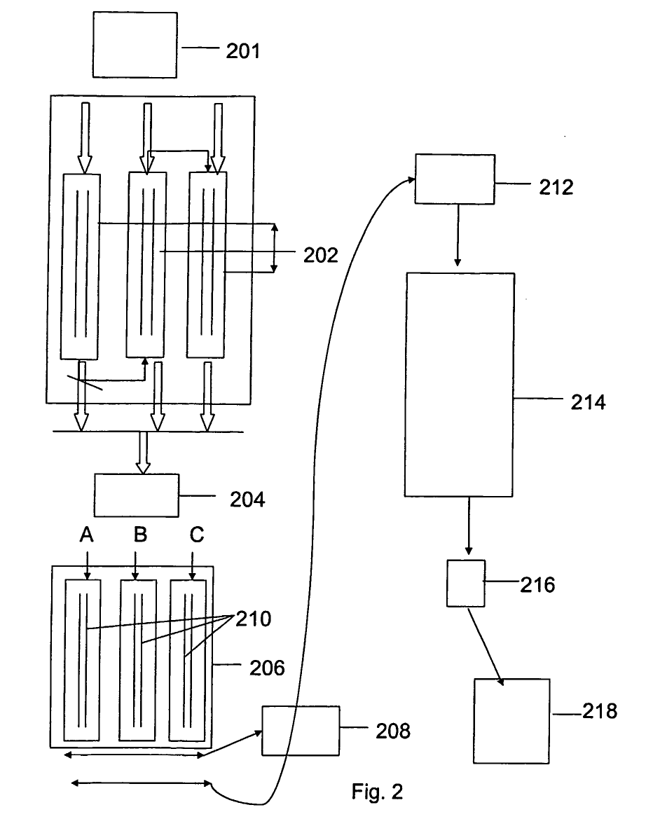

The inventive method comprises both direct and indirect methods of screening. The indirect method involves clearing, binding and elution cycles to screen large number of drug candidates. As shown in FIG. 2, the indirect method involves an application of a library of drug candidates 201 mixed in a dilution buffer to a series of pre-determined tissue sections 202 for pre-clearing purposes. These pre-determined tissue sections 202 typically contain tissue samples which are normal for the target disease state. It is contemplated within the scope of the invention these pre-determined tissue samples 202 may be obtained from human, mouse, rat or any other animal offering an appropriate clinical model.

The pre-determined tissue samples 202 may be frozen and/or fixed and may be embedded in paraffin or any other embedding medium as known to those skilled in the art. It is contemplated within the scope of this invention that tissue samples can be live tissue that are cultured upon a surface.

The application of the library of compounds 201 to these pre-determined tissue samples 202 allows for the elimination of those compounds binding to normal tissue. Those molecules not bound to normal tissue within the tissue cartridge are referred to as “flow-through” molecules 204. These flow-through molecules 204 are applied to a series of disease state tissue cartridges 206. The disease state tissue cartridges 206 contain tissue samples representative of the disease state of interest. It is contemplated within the scope of this invention that these disease state tissues include but are not limited to neoplasms, mesenchymal tumors, fibroma, fibrosarcoma, mesothelioma, leukemia, epithelial tumors, basal cell carcinoma, squamous cell carcinoma, adenocarcinoma, melanoma, seminoma, Alzheimer's, myasthenia gravis, diabetes mellitus, chronic inflammations, septic shock, Down Syndrome, Gaucher Disease, infectious diseases, Prion, HPV, HIV, Tuberculosis and Poliovirus.

Unbound molecules 208 are washed from the disease state tissue cartridges 206 using wash buffers. The unbound molecules 208 are eliminated as candidates. The wash buffers may be the same as the dilution buffers listed above or dilution buffers without the protein component. Other biological wash buffers known in the art may be used such as Tris Buffered Saline (TBS), pH 7.4, TBS with Tween 20 (0.1%), or PBS with Tween 20 (0.1%). It is contemplated within the scope of the invention that other biological buffers may be used such as MES, BIS-TRIS, ADA, ACES, PIPES, MOPSO, BES, MOPS, TES, HEPES, DIPSO, MOBS, TAPSO, TRIZMA, POPSO, TEA, TRICINE, BICINE and TAPS. (Sigma-Aldrich, St. Louis, Mo., Catalogue pages 1873, 2001).

Flow-through molecules 204 binding to disease state tissues are determined to be specifically bound drug candidates 210. Specifically bound drug candidates 210 are eluted by pre-determined elution buffers. These elution buffers include but are not limited to high pH buffers such as Ttriethylamine buffer (100 mM, pH 11.5-12.5), Glycine buffer (100 mM, pH 11), and low pH buffers such as Glycine buffer (100 mM, pH 1.8-2.7), HCl (0.1N). High salt buffers may also be used as elution buffers such as LiCl (5M, Phosphate buffer, pH 7.2), MgCl₂ (3.5M, Phosphate buffer, pH 7.2), NaCl ([1-5M). Additionally, low pH and high salt buffers may be used such as Glycine buffer (100 MM, 0.5M NaCl, pH 2.7), high salt and pH buffers such as NaCl (3M, Borate buffer, pH 9.0), detergents such as SDS (Sodium dodecyl sulfate, 0.1-1%), DOC (Sodium deoxycholate, 0.1-1%). Chelating agents are also contemplated within the scope of this invention for elution such as EDTA (50 mM Tris-acetate buffer, 150 mM NaCl, EDTA, 20 mM, pH 7.5). Dissociating agents are also contemplated such as Urea (2-6M) and Guanidine-HCL (2-8M). Chaotropic agents may also be used such as Thiocyanate (3M). Reducing agents such as DTT (5-50 mM) and organic solvents such as Dioxane (100%), Ethylene glycol, (50%, pH 11.5 or pH 8.0) can also be used to eluted bound compounds. Other compounds known in the art may be used to detach bound compounds from disease state tissue.

These eluted specifically bound drug candidates 212 are collected and separated using chromatography methods 214 known in the art. The separated compounds are biotinylated 216 (Pierce Biotechnology, Rockford Ill.) and identified using detection methods 218 as described in “Scorilas A and Diamandis EP (2000) Polyvinylamine-streptavidin complexes labeled with a europium chelator: A universal detection reagent for solid-phase time resolved fluorometric applications. Clinical Biochemistry 33(5):345-350”. Which is incorporated in its entirety by reference. Those specifically bound drug candidates 210, which are bound and then eluted, are indicative of their affinities to specific target tissues contained within the disease state tissue cartridges 206.

The eluted drug candidates' binding affinity and specificity may be further understood by additional cycles of clearing, binding and elution steps. It is contemplated within the scope of this invention that once identified the eluted compounds can be reapplied to cartridges containing normal tissue samples or no tissue samples for control purposes. It is further contemplated within the scope of this invention that eluted compounds can be applied to normal tissue from various organs to further define the specificity of the binding characteristics of the identified compounds.

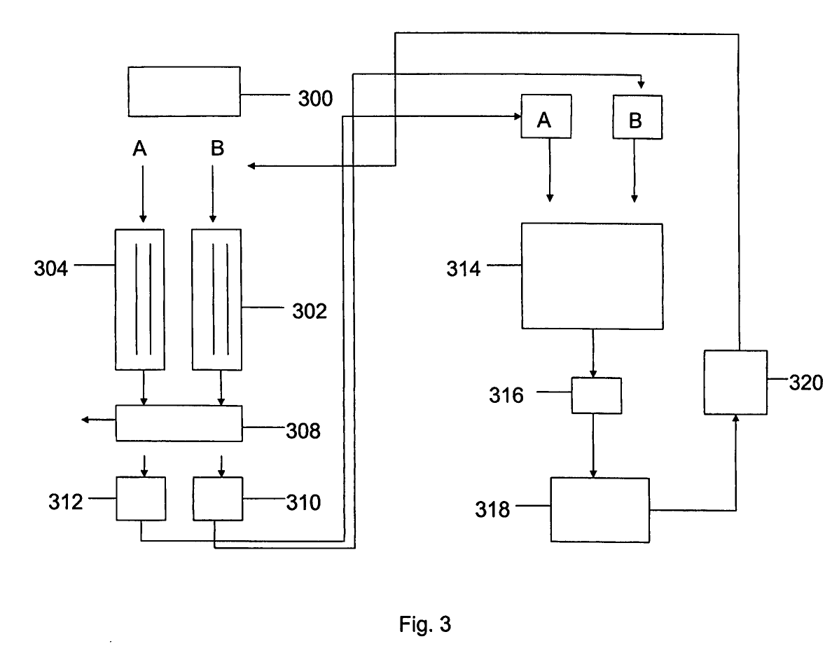

In an alternative illustrative embodiment a direct approach to the affinity of a compound to selected tissue samples may be assessed. The direct approach method, as shown in FIG. 3, screens drug libraries directly to designated tissues under designated high affinity, high stringent conditions. The direct method, unlike the indirect method, does not pre-clear a library of compounds 300 through normal tissue samples. The library of compounds 300 containing approximately 10,000 compounds is diluted with a dilution buffer and passed through tissue cartridges containing both disease state tissue 302 and non-disease state tissue 304.

The dilution buffers allow compounds within the library of compounds 300 to interact with tissue samples in conditions that mimic in-vivo conditions. Additionally, the dilution buffers allow the concentration of the library of compounds 300 to be adjusted to mimic blood concentration levels. These dilution buffers are composed of two main components a solution, and protein that are buffered to a specific pH. The solution may be composed of phosphate buffered saline (PBS), saline, balanced salt solution (BSS) or selected mixture of serum components (SMSC) (e.g. amino acids, vitamins, ions, metabolites, lipids, carbohydrates, etc). SMSC may be composed of certain components to reflect pathological conditions such as high glucose for diabetes. The protein may include blood, serum or plasma at approximately 0.1-50% (v/v). The dilution buffer's pH may reflect arterial blood (7.38-7.44) or venous blood (7.76-7.41) or venous serum/plasma (7.35-7.45). Under certain conditions where maximum protein effect is needed, dilution buffers may be composed of 100% blood, serum or plasma. Other dilution buffers known in the art may also be used.

The diluted library of compounds 300 are applied to a series of disease state tissue cartridges 302 and a series of non-disease state tissue cartridges 304. The disease state tissue cartridges 302 contain tissue samples having a disease state of interest. The non-disease state tissue cartridges 304 contain tissue samples containing normal tissue for the same body part as the disease state tissue. The applied compounds are incubated at approximately 37° C. for approximately 12 hours. Unbound molecules 308 are washed from the disease-state tissue cartridges 302 and non-disease state tissue cartridges 304 using wash buffers. The unbound molecules 308 from the disease state cartridges 302 are eliminated as candidates. Bound molecules 312 are eluted from disease state tissue cartridges 302 and non-disease state tissue cartridges 304. Specific disease state compounds are identified using comparative analysis and differences in binding profiles between disease state tissue cartridges 302 and non-disease state tissue cartridges 304.

The wash buffers may be the same as the dilution buffers listed above or the dilution buffers without the protein component. Other biological wash buffers known in the art may be used such as Tris Buffered Saline (TBS), pH 7.4, TBS with Tween 20 (0.1%), or PBS with Tween 20 (0.1% ). Molecules binding to disease state tissues are determined to be specifically bound drug candidates 312. Specifically bound drug candidates 312 are eluted by pre-determined elution buffers.

These elution buffers include but are not limited to high pH buffers such as Ttriethylamine buffer (100 mM, pH 11.5-12.5), Glycine buffer (100 mM, pH 11), and low pH buffers such as Glycine buffer (100 mM, pH 1.8-2.7), HCl (0.1N). Also high salt buffers may be used as elution buffers such as LiCl (SM, Phosphate buffer, pH 7.2), MgCl₂ (3.5M, Phosphate buffer, pH 7.2), NaCl ([1-5M). Additionally, low pH and high salt buffers may be used such as Glycine buffer (100 mM, 0.5M NaCl, pH 2.7), high salt and pH buffers such as NaCl (3M, Borate buffer, pH 9.0), detergents such as SDS (Sodium dodecyl sulfate, 0.1-1%), DOC (Sodium deoxycholate, 0.1-1%). Chelating agents are also contemplated within the scope of this invention for elution such as EDTA (50 mM Tris-acetate buffer, 150 mM NaCl, EDTA, 20 mM, pH 7.5). Dissociating agents are also contemplated such as Urea (2-6M) and Guanidine-HCL (2-8M). Chaotropic agents may also be used such as Thiocyanate (3M). Reducing agents such as DTT (5-50 mM) and organic solvents such as Dioxane (10%), Ethylene glycol, (50%, pH 11.5 or pH 8.0) can also be used to eluted bound compounds. Other compounds known in the art may be used to detach bound compounds from disease state tissue.

The eluted compounds 312 from the disease state tissue cartridges 302 and the bound compounds 310 from the non disease state tissue cartridges 304 are collected and applied to a 2D HPLC system 314 and fractionated according to Wagner, K., et al., (2000) Protein mapping by two-dimensional high performance liquid chromatography. J. Chromatography 893(2):293-305. This method is incorporated herein in its entirety. The fractions are collected and biotinylated (Pierce Biotechnology, Rockford Ill.) and presence or absence of compounds is detected with an ultra sensitive detection system 318 (USD) (Scorilas A et al.).

If too many peaks are detected indicating a high level of binding to tissue within the non-disease state tissue cartridges 304 the parameters of the assay can be changed. For example, the library of compounds can be reduced. Incubation temperature can be changed and the length of incubation time can be reduced to approximately 10-20 minutes. The parameters are repeatedly adjusted until there is no binding on tissue within the non-disease state tissue cartridges 304.

Likewise, if there is no binding of compounds detected on disease state tissue sections the library of compounds concentration can be increased or incubation temperature and/or time can be adjusted. The assay can be redone with changed parameters establishing a clear binding/elution profile for about 1-10% of applied compounds amount, resulting in about 1,000 identified compounds 320.

The identified compounds 320 are prepared similar to the above concentration. The compounds are applied to the non-disease state cartridges 304 and disease state tissue cartridges 302 prepared in the initial assay step. The mixture is incubated. Bound compounds are eluted and fractions are applied to a 2D HPLC system 314 and fractionated according to (Wagner, K., et al., (2000)). The fractions are collected and biotinylated (Pierce Biotechnology, Rockford Ill.) and identified using an ultra sensitive detection system 318 (Scorilas, A. et al.). Those compounds having affinity to non-disease state tissue sections are eliminated as candidates and those compounds having affinity to disease state tissue are retained and identified.

Compounds having complete affinity for disease state tissue sections are identified as final drug candidates. Each final drug candidate is applied at a given concentration within a vial containing 50% blood within a PBS buffer to a further set of tissue cartridges. This further set of tissue cartridges will be comprised of ten cartridges containing normal tissue and ten cartridges containing cancerous tissue. Additional tissue cartridges containing tissue sections from various body parts are also prepared. Two tissue cartridges of each body part are prepared. These body parts comprise normal liver sections, normal kidney sections, normal skeletal muscle sections, normal smooth muscle sections, normal lung sections, normal pancreas sections, normal cerebellum sections, normal cerebrum sections, normal lymph node sections and normal stomach sections. Additional normal sections may be included depending on the route of administration and excretion from the body.

The applied drug candidates are incubated at approximately 37° C. for approximately 12 hours. Bound compounds are eluted and detected as set forth above. Those compounds exhibiting binding upon any normal tissue sections are eliminated as drug candidates. Additionally, those compounds showing no binding upon disease state tissue are also eliminated. Those compounds exhibiting binding upon only disease state tissue sections are selected and further applied to additional tissue cartridges for validation.

### EXAMPLE 1

### In-Direct Application Method

In this first illustrative embodiment a library of drug candidates for breast cancer comprised of 10,000 peptides is assessed. The goal of the assessment using the indirect method is the identification of those compounds within the library that are specific for breast cancer. The expectations in this example are that normal breast tissue should not show any binding. The protocol for this example is a targeted half-life of twelve hours and a targeted blood concentration of approximately 0.1 μg/ml. The administration of the compound is via intra-muscular administration.

A peptide mixture is prepared by combining approximately 0.5 μg/ml of each peptide within a vial in a solution containing 50% blood in a PBS buffer. Ten vials each containing approximately one thousand peptides are prepared. Each vial sample is applied to 2D HPLC (Wagner, K., et al) and approximately 400 fractions are collected. The collected fractions are analyzed using MALDI-TOF mass spectroscopy and each peptide is identified. After identification, peptides are biotinylated (Pierce Biotechnology, Rockford, Ill.) and detection peak profiles are established using an ultra-sensitive detection system (USD) (Scorilas, A., et al). Each peptide's 2D HPLC profile and individual detection limits are established.

After the preparation and identification of peak profiles, all peptides within the library are applied to pre-clearance tissue cartridges containing those tissues that show nonspecific binding. Since the goal is to find those compounds having no binding upon normal breast sections and the targeted route of administration is via intramuscular administration, the pre-clearance tissue cartridges are comprised of ten cartridges having normal breast tissue and ten cartridges having normal skeletal muscle tissue. Also included within this pre-clearance step is ten cartridges having normal lymph node tissue due to their implication in metastatic breast cancer.

In the instant example the total tissue accessible is estimated to be about 10 mg for 30 cartridges (5 micron cut from a paraffin section generates approximately 0.4-0.8 mg±per tissue section). In each tissue cartridge there is one plate with 2 tissue sections providing approximately 24-48 mg±total tissue weight for 30 tissue cartridges. It is assume that there is an approximate accessibility of 40% of total tissue, which provides a range of 9.6 to 19.2 mg weight. It is assumed that the lower range of estimated weight: 10 mg is the total accessible weight.

The prepared peptides are applied to 30 tissue cartridges having normal tissue sections and the flow-through molecules are collected. The collected flow-through contains those molecules unbound to normal tissue sections contained within the 30 tissue cartridges. These flow-through molecules having no affinity to normal tissue sections are applied to designated positive and negative breast cancer sections. In the instant example ten tissue cartridges are prepared containing five tissue cartridges with normal breast tissues and five tissue cartridges with breast cancer tissue sections.

Flow-through molecules are applied to these ten tissue cartridges having both positive and negative tissue samples. The applied flow through molecules and tissue samples are incubated at about 37° C. for approximately 12 hours. After incubation, unbound peptides are washed with PBS until a normal baseline is observed. The wash step is monitored with an on-line detection system such as UV absorption. Other on-line detection systems known in the art may be used. Bound peptides are eluted with a glycine buffer (pH 2.2).

The eluted peptides from each tissue cartridge are applied and fractionated using 2D [IE/RP] HPLC procedure (Wagner, K., et al.). The fractions are biotinalyated (Pierce Biotechnology, Rockford Ill.) and detected using an ultra sensitive detection method (Scorilas, A., et al.). The fractions are then analyzed based upon the detection. If there are too many peaks and lots of binding on normal breast sections then the stringency of the assay is changed. These changes may include dilution of the flow through and a shorter incubation time. The goal of these changes is to observe no binding upon normal breast sections. It is possible that high binding on normal breast sections may come from the blood reservoir part of the flow-through sample. To prevent this from occurring a pre-clearance step against blood may be included.

If no binding and therefore no peptides are detected on breast cancer sections then the concentration of peptides are increased and incubation is changed. The pre-clearance step would then be repeated with changed concentrations and incubation.

Once eluted drug candidates are identified, a peptide mixture is prepared and applied to a second series of tissue cartridges. This mixture is applied to a second series of tissue cartridges having ten tissue cartridges with normal breast tissue and ten cartridges with breast cancer tissue. The mixture is incubated at 37° C. for approximately 12 hours. Bound peptides are eluted and fractions are collected and identified and analyzed. Those compounds showing binding in all breast cancer tissue cartridges are selected for further confirmation as drug candidates.

Each drug candidate is applied to a further set of confirmation tissue cartridges with breast and other tissue sections. This set of confirmation tissue cartridges will once again contain ten cartridges containing normal breast tissue and ten cartridges containing cancerous breast tissue. The confirmation tissue cartridges will also be comprised of normal tissue sections from various body parts. Two tissue cartridges of each body part is prepared. These body parts comprise normal liver sections, normal kidney sections, normal skeletal muscle sections, normal smooth muscle sections, normal lung sections, normal pancreas sections, normal cerebellum sections, normal cerebrum sections, normal lymph node sections and normal stomach sections. Additional normal sections may be included depending on the route of administration and excretion from the body.

The drug candidates are applied to this series of confirmation tissue cartridges and incubated at approximately 37° C. for approximately 12 hours. Bound peptides are eluted and detected as set forth above. Those compounds exhibiting binding upon any normal tissue sections are eliminated as drug candidates. Additionally, those compounds showing no binding upon breast cancer tissue are also eliminated. Compounds exhibiting binding to only breast cancer sections are selected and further applied to additional tissue cartridges for validation.

### EXAMPLE 2

### One Step Direct Application Method

In an alternative illustrative embodiment a library of drug candidates for breast cancer comprised of 10,000 peptides are assessed. The goal of the assessment using a direct method is the identification of those compounds within the library that are specific for breast cancer.

A peptide mixture containing 10,000 peptides is prepared by combining approximately 0.1 μg/ml of each peptide within a vial containing 50% blood within a PBS buffer. The peptide mixture is applied to 2D HPLC (Wagner, K., et al.) and approximately 400 fractions are collected. The collected fractions are analyzed using MALDI-TOF mass spectroscopy and each peptide is identified. After identification, peptides are biotinylated (Pierce Biotechnology, Rockford Ill.) and detection peak profiles are established using an ultra sensitive detection method (Scorilas, A, et al.). Each peptide's 2D HPLC profile and individual detection limits are established.

Twenty tissue cartridge are prepared. Ten cartridges contain breast cancer tissue sections and ten cartridges contain normal breast sections. The peptide mixture is applied to each tissue cartridge for approximately one hour at 37° C. The un-bound peptides are then washed with PBS buffer until normal baseline is observed. The wash step is monitored with an on-line detection system such as UV absorption. Other detection systems known in the art may be used.

Bound peptides are then eluted with glycine buffer (pH 2.2). Other buffers known within the art may be used to elute the bound peptides. Eluted peptides from each tissue cartridge are collected and applied to a 2D HPLC system (Wagner, K., et al.) and fractionated. The fractions are collected and peptides are biotinylated (Pierce Biotechnology, Rockford Ill.). The presence or absence of peptide are detected with an ultra sensitive detection (Scorilas, A. et al.)

If too many peaks are detected indicating a high level of binding on normal breast sections the parameters of the assay are changed. The peptide concentration is reduced from about 0.1 ug/ml to about 0.01 ug/ml. Incubation temperature is change to about 45° C. and the length of incubation is reduced to approximately 10-20 minutes. The parameters are adjusted until there is no binding on normal breast sections

Likewise, if there is no binding of peptides detected on breast cancer sections the peptide concentration is increased or the incubation temperature or time is adjusted. The assay is redone with changed parameters establishing clear binding/elution profile for about 1-10% of applied peptide amount resulting in about 1,000 identified peptides.

A peptide mixture containing the identified 1,000 peptides is prepared similar to above in a concentration of approximately 0.1 μg/ml of each peptide within a vial containing 50% blood within a PBS buffer. The peptide mixture is applied to the 20 Tissue-Cartridges prepared in the initial assay step. The mixture is incubated. Bound peptides are eluted and are applied to a 2D HPLC system (Wagner, K., et al.). The fractions are collected and peptides are biotinylated (Pierce Biotechnology, Rockford Ill.) and identified using an ultra sensitive detection system (Scorilas, A. et al.). Those peptides having affinity to normal breast tissue sections are eliminated as candidates and those peptides having affinity to cancerous breast tissue are identified.

Peptides having complete affinity for breast cancer are identified as final drug candidates. Each final drug candidate is applied at a concentration of approximately 0.1 μg/ml of each peptide within a vial containing 50% blood within a PBS buffer to a further set of tissue cartridges with breast and other tissue sections. This further set of tissue cartridges will be comprised often cartridges containing normal breast tissue and ten cartridges containing cancerous breast tissue. Additional tissue cartridges containing tissue sections from various body parts are also prepared. Two tissue cartridges of each body part are prepared. These body parts comprise normal liver sections, normal kidney sections, normal skeletal muscle sections, normal smooth muscle sections, normal lung sections, normal pancreas sections, normal cerebellum sections, normal cerebrum sections, normal lymph node sections and normal stomach sections. Additional normal sections may be included depending on the route of administration and excretion from the body.

The applied drug candidates are incubated at approximately 37° C. for approximately 12 hours. Bound peptides are eluted and detected as set forth above. Compounds exhibiting binding to any normal tissue sections are eliminated as drug candidates. Additionally, compounds showing no binding to breast cancer tissue are also eliminated. Compounds exhibiting binding to only breast cancer sections are selected and further applied to additional tissue cartridges for validation.

While the invention has been described in connection with specific illustrative embodiments thereof, it will be understood that it is capable of further modifications and this application is intended to cover any variations, uses, or alterations of the invention. In general, the principles of the invention and including such departures from the present disclosure as come within known or customary practice within the art to which the invention pertains and as may be applied to the essential features hereinbefore set forth and as follows in the scope of the appended claims.

Various other changes, omissions and additions in the form and detail of the present invention may be made therein without departing from the spirit and scope of the invention. Therefore, the above description should not be construed as limiting, but merely as exemplification of the various embodiments.

What is claimed is:

1. A method for screening drug candidates comprising:
providing a library of compounds;
preparing tissue sections, wherein said tissue sections can be disease state tissues sections and non-disease state tissue sections;
applying said library of compounds to said tissue sections wherein said compounds having affinity for said tissue sections bind to said tissue sections;
collecting flow-through compounds from said tissue sections;
washing unbound compounds from said tissue sections;
eluting said bound compounds from said tissue sections;
fractionating said eluted compounds; and
identifying said eluted compounds.

2. The method of claim 1 wherein said library of compounds are selected from the group consisting of peptides, proteins, antibodies, nucleic acids, carbohydrates, lipids, organic molecules and inorganic molecules.

3. The method of claim 1 wherein said fractionating is done with ion-exchange high pressure liquid chromatography.

4. The method of claim 1 wherein said fractionating is done with ion-exchange chromatography.

5. The method of claim 1 wherein said fractionating method is size exclusion high pressure liquid chromatography.

6. The method of claim 1 wherein said fractionating is done with gel filtration chromatography.

7. The method of claim 1 wherein said fractionating method is reverse phase high pressure liquid chromatography.

8. The method of claim 1 wherein said fractionating method is reverse phase liquid chromatography.

9. The method of claim 1 wherein said fractionating method is affinity chromatography.

10. The method of claim 1 wherein said fractionating method is hydrophobic interaction chromatography.

11. The method of claim 1 wherein said fractionating method is flow cytometry.

12. The method of claim 1 wherein said fractionating method is electrophoresis.

13. The method of claim 1 wherein said fractionating method is capillary electrophoresis.

14. The method of claim 1 wherein said detection method is immunoaffinity capillary electrophoresis.

15. The method of claim 1 wherein said detection method is biotin-streptavidin detection with a reporter structure.

16. The method of claim 1 wherein said detection method is PCR (polymerase chain reaction).

17. The method of claim 1 wherein said unbound compounds from said non-disease state tissue are further applied to disease state tissue.

18. The method of claim 1 wherein said library of compounds are diluted in pre-selected dilution buffers before said tissue application.

19. The method of claim 1 wherein said non disease state tissue sections are selected from a group consisting of normal breast sections, normal liver sections, normal kidney sections, normal skeletal muscle sections, normal smooth muscle sections, normal lung sections, normal pancreas sections, normal cerebellum sections, normal cerebrum sections, normal lymph node sections, normal stomach sections.

20. The method of claim 1 wherein said disease state tissue sections are selected from a group consisting of cancerous breast sections, cancerous liver sections, cancerous kidney sections, cancerous skeletal muscle sections, cancerous smooth muscle sections, cancerous lung sections, cancerous pancreas sections, cancerous cerebellum sections, cancerous cerebrum sections, cancerous lymph node sections, cancerous stomach sections.

21. The method of claim 1 wherein said disease state tissue are selected from the group consisting of neoplasms, mesenchymal tumors, fibroma, fibrosarcoma, mesothelioma, leukemia, epithelial tumors, basal cell carcinoma, squamous cell carcinoma, adenocarcinoma, melanoma, seminoma, Alzheimer's, myasthenia gravis, diabetes mellitus, chronic inflammations, septic shock, Down Syndrome, Gaucher Disease, infectious diseases, Prion, HPV, HIV, Tuberculosis and Poliovirus.

22. The method of claim 1 wherein said tissue sections are paraffin embedded tissues sections.

23. The method of claim 1 wherein said tissue sections are frozen tissue sections.

24. The method of claim 1 wherein said tissue sections are live tissue sections on a solid support.

25. The method of claim 1 wherein said un-bound or nonspecifically bound compounds are washed using a buffer selected from the group consisting of TBS [Tris Buffered Saline, pH 7.4], TBS with Tween 20 (0.1%) and PBS with Tween 20 (0.15).

26. The method of claim 1 wherein said un-bound compounds are washed using a biological buffer selected from the group consisting of MES, BIS-TRIS, ADA, ACES, PIPES, MOPSO, BES, MOPS, TES, HEPES, DIPSO, MOBS, TAPSO, TRIZMA, POPSO, TEA, TRICINE, BICINE and TAPS.

27. The method of claim 1 wherein said bound compounds are eluted using an elution compounds selected from the group consisting of high pH buffers, low pH buffers, high salt buffers, low pH and high salt buffers, high salt and pH buffers, detergents, chelating agents, dissociating agents, chaotropic agents, reducing agents and organic solvents.

28. The method of claim 1 wherein said un-bound compounds are washed from said tissue sections until a normal baseline is observed.

29. The method of claim 1 wherein said library of compounds are applied to non disease state tissue and disease state tissue in parallel.

30. The method of claim 1 wherein said library of compound are applied only to non disease state tissue and said non disease state tissue acts as a filter.

31. The method of claim 1 wherein said library of compounds are applied only to disease state tissue.

32. The method of claim 1 wherein said tissue sections are human.

33. The method of claim 1 wherein said tissue sections are animal.

34. A tissue cartridge for conducting tissue based affinity chromatography comprising:
a sealable tissue chamber said sealable tissue chamber having a first plate and a second plate, wherein said first and second plate receive tissue samples;
a heating element contained within said sealable tissue chamber
said sealable tissue chamber having an inlet port and an outlet port, said outlet port being in fluid communication to an outlet channel said outlet channel having an outlet valve, said inlet port being in fluid communication to an inlet channel said inlet channel having an inlet valve said inlet channel having a fluidic pump disposed within;
a connecting channel providing for fluid communication between said inlet channel and said outlet channel, wherein said connecting channel has a first connecting valve and a second connecting valve;

35. The tissue cartridge of claim 34, wherein said fluidic pump recycles fluid through said tissue cartridge.

36. The tissue cartridge of claim 34, wherein said heating element is a printed resistive ink applied to said sealable tissue chamber.

37. The tissue cartridge of claim 34, wherein said heating element is a resistive heating element contained within said sealable tissue chamber.

38. The tissue cartridge of claim 34, wherein said first plate and said second plate are standard glass microscope slides.

39. The tissue cartridge of claim 34, wherein said sealable tissue chamber is made from plastic selected from the group consisting of poly(methyl methacrylate), acetonitrile-butadiene-styrene, polycarbonate, polyethylene, polystyrene, polyolefins, polypropylene, polyphenylene sulphide, PEEK, and metallocene.

40. The tissue cartridge of claim 34, wherein said channels are made from plastic selected from the group consisting of poly(methyl methacrylate), acetonitrile-butadiene-styrene, polycarbonate, polyethylene, polystyrene, polyolefins, polypropylene, polyphenylene sulphide, PEEK, and metallocene.

41. The tissue cartridge of claim 34 wherein said heating element is connected to a variable control sensor.

42. The tissue cartridge of claim 34 wherein said sealable tissue chamber further contains a thermocouple that controls said heating element.
